(19) United States
(12) Patent Application Publication
(10) Pub. No.: US 2025/0197398 A1
(43) Pub. Date: Jun. 19, 2025

(54) Varenicline Related Compounds and Methods for Treating Diseases and Conditions Including Tobacco Use Disorder

(71) Applicant: SpaceRX LLC, Reston, VA (US)

(72) Inventors: Peter H. LEE, Reston, VA (US); John Nicholas BETLEY, Bala Cynwyd, PA (US)

(21) Appl. No.: 19/066,670

(22) Filed: Feb. 28, 2025

Related U.S. Application Data
(63) Continuation of application No. PCT/US2023/073315, filed on Sep. 1, 2023.
(60) Provisional application No. 63/403,529, filed on Sep. 2, 2022.

(51) Int. Cl. C07D 471/18; A61K 31/55

(52) U.S. Cl. CPC C07D 471/18; A61K 31/55

(57) ABSTRACT

Provided herein are compounds, pharmaceutically acceptable salts, pharmaceutical compositions containing these compounds or pharmaceutically acceptable salts thereof, kits containing these compounds or pharmaceutically acceptable salts thereof, methods of making these compounds or pharmaceutically acceptable salts thereof, and methods of treating and preventing diseases and conditions using these compounds or pharmaceutically acceptable salts thereof.

### CROSS-REFERENCE TO RELATED APPLICATIONS

This application is a continuation of PCT/US2023/073315, filed Sep. 1, 2023, which claims the benefit of U.S. Provisional Application No. 63/403,529, filed Sep. 2, 2022, the entire contents of each of which are incorporated herein by reference in their entireties.

### SUMMARY

In some embodiments, provided herein are a pharmaceutically acceptable salt of formula I:

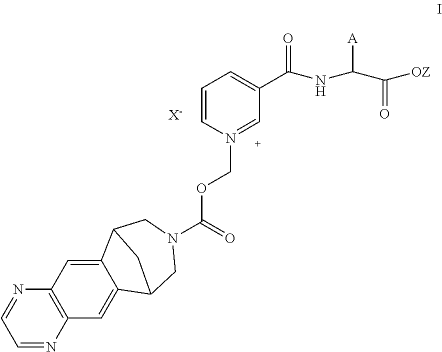

wherein, independently, X⁻ can be a pharmaceutically acceptable anion, A can be a C1-C10 alkyl or branched alkyl group bearing a hydroxyl group, or any amino acid side chain, and Z can be a hydrogen or a pharmaceutically acceptable cation, or the following fragment:

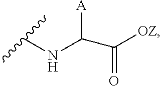

of the pharmaceutically acceptable salt of the compound of formula I and can be D, L, or racemic or achiral; for example, a fragment can be a fragment of any of the following amino acids: Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine. Valine, or, Selenocysteine; or a salt of any of these.

In some embodiments is provided a pharmaceutically acceptable salt of formula 1A:

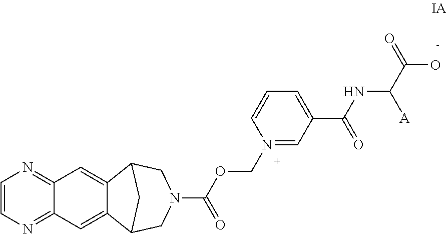

wherein A is a C1-C10 alkyl or branched alkyl group bearing a hydroxyl group or a C1-C10 straight chain or branched chain alkyl group bearing a carboxylic acid group or a pharmaceutically acceptable salt thereof.

In keeping with the above, the pharmaceutically acceptable salt of the compound of formula I can be a pharmaceutically acceptable salt of formula IB:

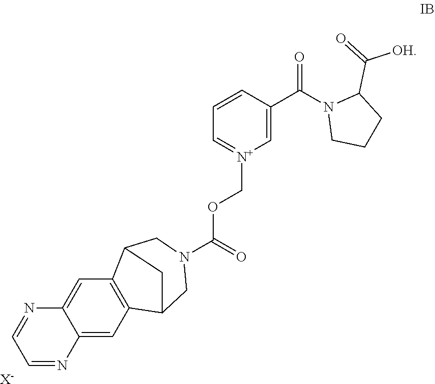

In some embodiments, in the pharmaceutically acceptable salt of formula I or formula IB, X⁻ can be an anion selected from the group consisting of: chloride, bromide, sulphate, phosphate, acid phosphate, acetate, maleate, fumarate, lactate, tartrate, citrate, gluconate, and besylate. In some embodiments, in the pharmaceutically acceptable salt of the compound of formula I, Z can by hydrogen (H). In some embodiments, in the pharmaceutically acceptable salt of the compound of formula I, Z can be a pharmaceutically acceptable organic or inorganic cation.

In some embodiments, the pharmaceutically acceptable salt of formula I can have the structure of formula II:

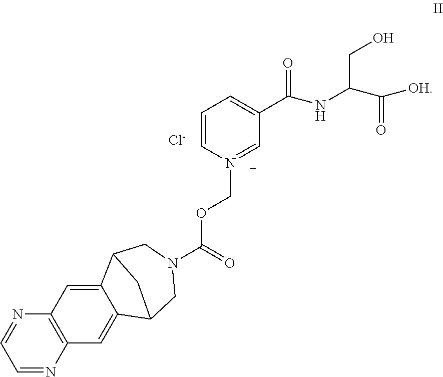

In some embodiments, the pharmaceutically acceptable salt of formula I can have the structure of formula III:

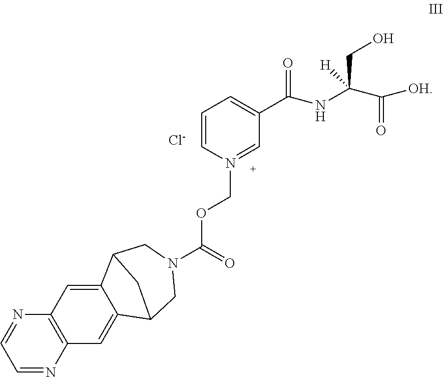

In some embodiments is provided a pharmaceutically acceptable salt of formula IV:

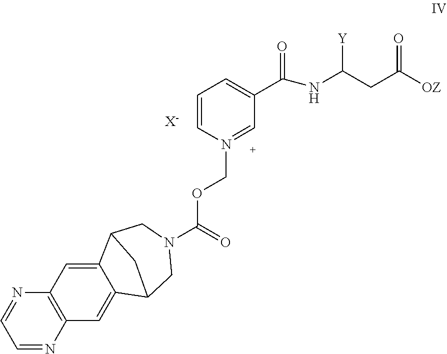

wherein, independently, X can be a pharmaceutically acceptable anion, Y can be a C1-C10: straight chain or branched chain alkyl group bearing a carboxylic acid group or a pharmaceutically acceptable salt thereof, and Z can be hydrogen or a pharmaceutically acceptable cation.

In some embodiments, in the pharmaceutically acceptable salt of formula IV, X− can be an anion selected from the group consisting of: chloride, bromide, sulphate, phosphate, acid phosphate, acetate, maleate, fumarate, lactate, tartrate, citrate, gluconate, and besylate. In some embodiments, in the pharmaceutically acceptable salt of the compound of formula IV, Z can be hydrogen (H). In some embodiments, in the pharmaceutically acceptable salt of the compound of formula IV, Z can be a pharmaceutically acceptable organic or inorganic cation.

In some embodiments, the pharmaceutically acceptable salt of formula IV can have the structure of formula V:

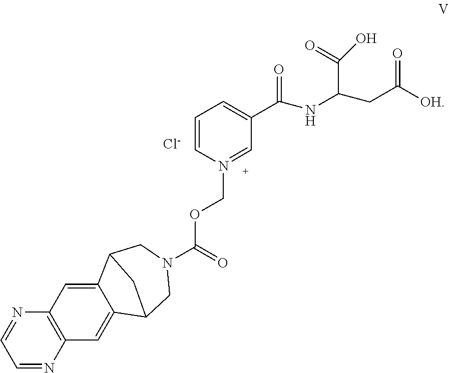

In some embodiments, the pharmaceutically acceptable salt of formula VI can have the structure of formula VI:

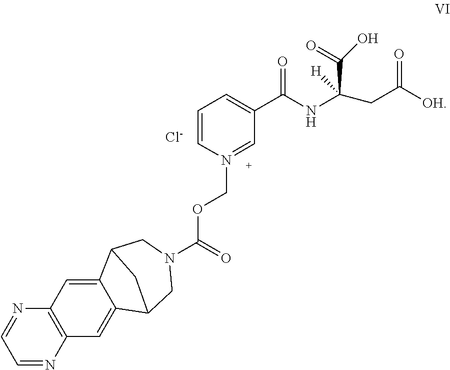

In some embodiments is provided a compound of formula VII or a pharmaceutically acceptable salt thereof:

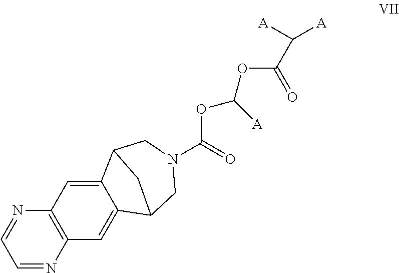

wherein each A, can be independently, a C1-C5 straight chain or branched chain methyl group.

In some embodiments, in a compound or pharmaceutically acceptable salt thereof of formula VII, each A can be, independently, methyl.

In some embodiments is provided a compound of formula VIII or a pharmaceutically acceptable salt thereof:

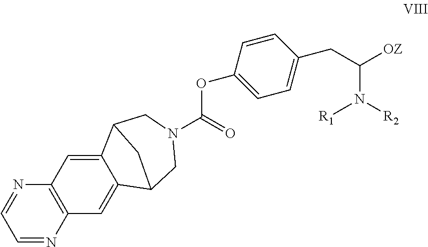

wherein R¹ and R² can be independently, hydrogen (H) or a C1-C5 branched or straight chain alkyl, or R¹ and R² together with the nitrogen atom to which they are attached can form a 5, 6, or 7 membered ring, and Z can be hydrogen (H) or a pharmaceutically acceptable cation.

In some embodiments, in a compound of formula VIII or a pharmaceutically acceptable salt thereof, R¹ and R² and Z can be hydrogen.

In some embodiments is provided a pharmaceutical composition comprising a pharmaceutically acceptable: salt or compound of formulae I through VIII and a pharmaceutically acceptable: excipient, diluent, carrier, or any combination thereof. In some embodiments, the pharmaceutical composition can be in unit dose form.

In some embodiments is provided method of treating or preventing a disease or condition in a subject or subject in need thereof, who can be a human subject or a human subject in need thereof, which can be a human subject in need thereof, comprising administering a pharmaceutically acceptable salt or a compound of formulae I through VIII, or a pharmaceutical composition comprising one or more of these and a pharmaceutically acceptable: carrier, excipient, diluent, or any combination of these; which can be in unit dose form, to the subject, human subject, subject in need thereof, or human subject in need thereof, in an effective or therapeutically effective amount sufficient to treat the disease or condition. In some embodiments, the disease or condition can be tobacco use disorder, nicotine use disorder, smoking, Parkinson's disease, or any combination thereof. In some embodiments, the disease or condition comprises tobacco use disorder. In some embodiments, the administering can be once, twice, or three times per day; or one every other day; or once every third day. In some embodiments, the administering can be conducted for a period of a day, a week, a month, 6 months, a year, two years, three years, four years, five years, for life, or as needed. In some embodiments, the human or human in need thereof can be a male or a female. In some embodiments, the human or human in need thereof can be age 18 or older. In some embodiments, the administering can be oral or inhaled or intranasal or subcutaneous or intramuscular or intravenous administrating. In some embodiments, any pharmaceutically acceptable salt or compound herein, including the pharmaceutically acceptable salt or the compound of formulae I through VIII can be administered in a dosage amount ranging from about 1 mg to about 20 mg, or about 1 mg to about 15 mg, or about 1 mg to about 10 mg, or about 1 mg to about 5 mg, or about 1 mg to about 3 mg. In some embodiments, any pharmaceutically acceptable salt or compound herein, including the pharmaceutically acceptable salt or the compound of formulae I through VIII, can be administered in a dosage amount ranging from about 0.00001 mg to about 10,000 mg per kg where mg is mg of pharmaceutically acceptable salt or compound and kg is body weight of the subject. For example, these can be administered in about: 0.001 mg per kg, 0.01 mg per kg, 0.1 mg per kg, 1 mg per kg, 10 mg per kg, 100 mg per kg,

In some embodiments is provided a kit comprising a pharmaceutically acceptable salt or compound of formulae I through VIII, or pharmaceutical composition containing one or more of these, and a container.

In some embodiments is provided a method of making the kit, comprising placing a pharmaceutically acceptable salt or compound of formulae I through formulae VIII, or any pharmaceutical composition containing these, in the container.

In some embodiments is provided methods of making the compounds or pharmaceutically acceptable salts thereof of formulae I through VIII, and also is provided intermediate compounds and their salts useful for making these and methods of making these.

The summary is intended to provide exemplary embodiments herein and is in no way intended to be otherwise limiting.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1B are a schematic and a response curve illustrating the monitoring of striatal dopamine in the awake behaving animal in response to varenicline. FIG. 1A shows the fiber photometry system and fiber placement used to monitor DA-induced fluorescent changes in awake, behaving mice and the representative image shows GPCR-activation-based-DA (GRAB-DA) and fiber placement in the striatum. Scale bar, 200 μm. FIG. 1B shows striatal dopamine responses preceding and following an intraperitoneal (IP) administration of nicotine.

FIG. 2A-FIG. 2C are bar graphs and a response curve illustrating the striatal dopamine responses to nicotine. FIG. 2A shows dopamine responses (mean for 30 minutes following nicotine administration) in animals pretreated with either vehicle, varenicline (1.5 and 4.5 mg/kg (mpk)), Servarenicline Chloride, Varenicline-Encarbil, Varenicline-CBT, or Varenicline Glycinamide. FIGS. 2B-2C show exemplary traces and quantification of striatal dopamine responses preceding and following an IP administration of nicotine. Mice were pretreated either with Varenicline (light grey) or Varenicline-Encarbil (dark grey).

FIG. 3A-FIG. 3B are a bar graph and a response curve illustrating the striatal dopamine responses to oral compound administration. FIG. 3A shows Dopamine responses (mean for 30 minutes following compound administration) in animals given with either vehicle, varenicline (1.5 and 4.5 mpk), Servarenicline Chloride, Varenicline-Encarbil, Varenicline-CBT, or Varenicline Glycinamide. FIG. 3B shows Temporal dynamics of Dopamine responses in animals given with either vehicle, varenicline (1.5 and 4.5 mpk), Servarenicline Chloride, Varenicline-Encarbil, Varenicline-CBT, or Varenicline Glycinamide.

FIG. 4 is a schematic illustrating approaches to stabilizing a dopamine response. FIG. 4 shows a dopamine response to treatment with a smoking cessation product or vehicle and the subsequent dopamine response to a dose of nicotine. Note how an ideal “prodrug” stabilizes the dopamine response throughout the day.

FIG. 5 is a bar graph illustrating open field responses to oral administration of compounds. FIG. 5 shows time spent in center of an open field test in animals given with either vehicle, varenicline (1.5 and 4.5 mpk), Servarenicline Chloride, Varenicline-Encarbil, Varenicline-CBT, or Varenicline Glycinamide.

### DETAILED DESCRIPTION OF THE INVENTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Herein, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be endometriosis, tobacco use disorder, nicotine use disorder, or smoking. In some cases, the subject may not be necessarily diagnosed or suspected of being at high risk for the disease.

The term “in vivo” can be used to describe an event that takes place in a subject's body.

The term “ex vivo” can be used to describe an event that takes place outside of a subject's body. An “ex vivo” assay may not be performed on a subject. Rather, it can be performed upon a sample separate from a subject. An example of an “ex vivo” assay performed on a sample can be an “in vitro” assay.

The term “in vitro” can be used to describe an event that takes place contained in a container for holding laboratory reagent such that it can be separated from the living biological source organism from which the material may be obtained. In vitro assays can encompass cell-based assays in which cells alive or dead are employed. In vitro assays can also encompass a cell-free assay in which no intact cells are employed.

As used herein, the term, ‘about’ a number, can refer to that number plus or minus 10% f that number. The term, ‘about’ a range, can refer to that range minus 10% f its lowest value and plus 10% f its greatest value.

As used herein, the terms “treatment” or “treating” can refer to a pharmaceutical or other intervention regimen for obtaining beneficial or desired results in the recipient. Beneficial or desired results can include but are not limited to a therapeutic benefit and/or a prophylactic benefit. A therapeutic benefit may refer to eradication or amelioration of one or more symptoms or of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “unit dose” or “dosage form” can be used interchangeably and can be meant to refer to pharmaceutical drug products in the form in which they are marketed for use, with a specific mixture of active ingredients and inactive components or excipients, in a particular configuration, and apportioned into a particular dose to be delivered. The term “unit dose” can also sometimes encompass non-reusable packaging, although the U.S. Food and Drug Administration (FDA) distinguishes between unit dose “packaging” or “dispensing”. More than one unit dose can refer to distinct pharmaceutical drug products packaged together, or to a single pharmaceutical drug product containing multiple drugs and/or doses. The term “unit dose” can also sometimes refer to the particles comprising a pharmaceutical composition, and to any mixtures involved. Types of unit doses may vary with the route of administration for drug delivery, and the substance(s) being delivered. A solid unit dose can be the solid form of a dose of a chemical compound used as a pharmaceutically acceptable drug or medication intended for administration or consumption.

As used herein, a “dose” can refer to a measured quantity of a therapeutic agent to be taken at one time.

As used herein, “pharmaceutically acceptable salt” can refer to pharmaceutical drug molecules, which may be formed as a weak acid or base, chemically made into their salt forms, for example, as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, the terms “effective amount” or “therapeutically effective amount” of a drug used to treat a disease can be an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose.

The term “substantially” or “essentially” can refer to a qualitative condition that exhibits an entire or nearly total range or degree of a feature or characteristic of interest. In some cases, substantially can refer to a pain or dopamine response level that varies from a mean or median pain or dopamine response level by about plus or minus: 1%, 2%, 3%, 4%, 5%, 6%, 7% 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. For example, substantially can refer to: 70%, 75%, 80%, 85%, 90%, 95%, 99%, or 100% reduced pain or dopamine response. In some cases, substantially can refer to at least about: 70%, 75%, 80%, 85%, 90%, 95%, 99%, or 100% f the total range or degree of a feature or characteristic of interest.

### Overview

Diseases or conditions such as tobacco use disorder, nicotine use disorder, smoking, and Parkinson's disease are damaging to individuals having these diseases. Because they (e.g., tobacco use disorder and nicotine use disorder) involve drug seeking or addictive behavior, molecules or pharmaceutically acceptable salts thereof can be useful in helping individuals to reduce or cease using tobacco or nicotine, in any and all forms, including but not limited to smoking and snuff.

In some embodiments, provided herein are a pharmaceutically acceptable salt of formula I:

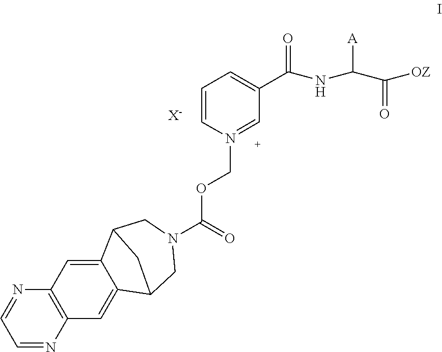

wherein, independently, X⁻ can be a pharmaceutically acceptable anion, A can be a C1-C10 alkyl or branched alkyl group bearing a hydroxyl group, and Z can be a hydrogen or a pharmaceutically acceptable cation.

In any compound or pharmaceutically acceptable salt herein containing one or more alkyl or branched alkyl groups, each alkyl or branched alkyl group can be, for example, independently, a methyl group, and ethyl group, a propyl group, an isopropyl group, a butyl group, a sec-butyl group, a tert-butyl group, an isobutyl group, a pentyl group, a hexyl group, a heptyl group, octyl group, a nonyl group, or a decyl group.

In some embodiments, in any salt herein, including in the pharmaceutically acceptable salt of formula I, the anion and/or X⁻ can be an anion selected from the group consisting of: chloride, bromide, iodide, sulphate, phosphate, acid phosphate, acetate, maleate, fumarate, lactate, tartrate, citrate, gluconate, and besylate. In some embodiments, in the pharmaceutically acceptable salt of the compound of formula I, Z can be hydrogen (H). In some embodiments, in the pharmaceutically acceptable salt of formula I, Z can be a pharmaceutically acceptable organic or inorganic cation. In some embodiments, the inorganic cation can be a sodium cation, a lithium cation, or a potassium cation. A magnesium cation—in some cases—the inorganic cation is associated with two molecules or pharmaceutically acceptable salts herein when the cation is a magnesium cation or calcium cation and bears a plus two charge, a lithium cation, a calcium cation—in some cases associated with two molecules or pharmaceutically acceptable salts herein when the calcium cation bears a plus two charge, or a potassium cation.

In some embodiments is provided a pharmaceutically acceptable salt of formula 1A:

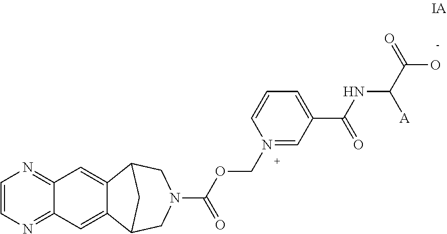

wherein A is a C1-C10 alkyl or branched alkyl group bearing a hydroxyl group or a C1-C10 straight chain or branched chain alkyl group bearing a carboxylic acid group or a pharmaceutically acceptable salt thereof.

In keeping with the above, the pharmaceutically acceptable salt of the compound of formula I can be a pharmaceutically acceptable salt of formula IB:

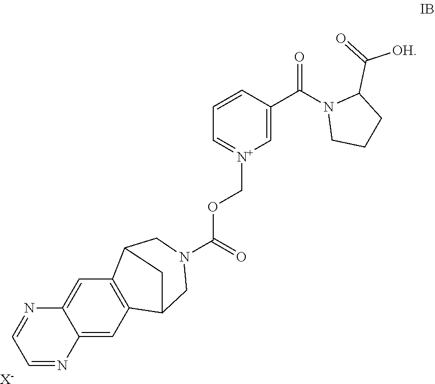

In some embodiments, in the pharmaceutically acceptable salt of formula I or formula IB, X⁻ can be an anion selected from the group consisting of: chloride, bromide, sulphate, phosphate, acid phosphate, acetate, maleate, fumarate, lactate, tartrate, citrate, gluconate, and besylate. In some embodiments, in the pharmaceutically acceptable salt of formula I, Z can by hydrogen (H). In some embodiments, in the pharmaceutically acceptable salt of formula I, Z can be a pharmaceutically acceptable organic or inorganic cation.

In some embodiments, the pharmaceutically acceptable salt of formula I can have the structure of formula II:

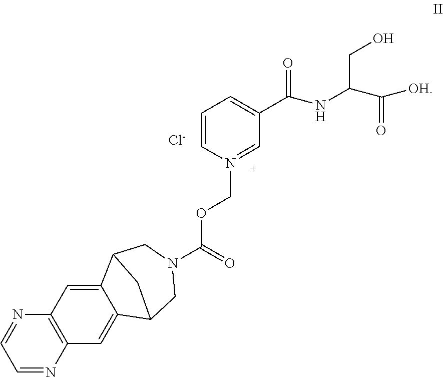

In some embodiments, the pharmaceutically acceptable salt of formula I can have the structure of formula III:

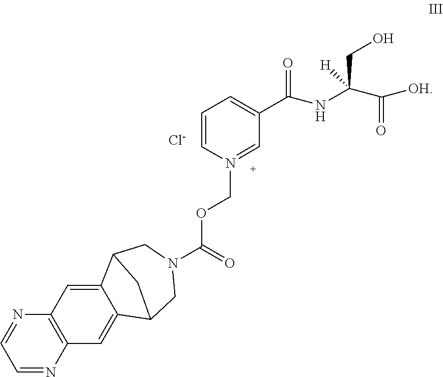

In some embodiments is provided a pharmaceutically acceptable salt of formula IV:

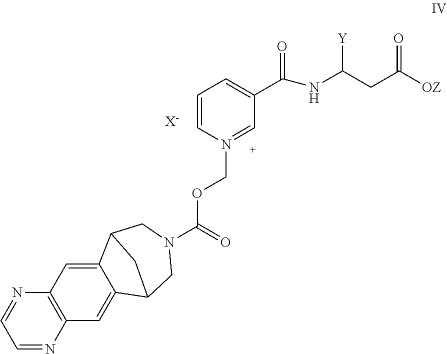

wherein, independently, X can be a pharmaceutically acceptable anion, Y can be a C1-C10: straight chain or branched chain alkyl group bearing a carboxylic acid group or a pharmaceutically acceptable salt thereof, and Z can be hydrogen or a pharmaceutically acceptable cation.

Any pharmaceutically acceptable salt or compound herein can be in the form, as applicable, of an enantiomer, a diastereomer, a racemic mixture of enantiomers, or a mixture of diastereomers, When any compound or pharmaceutically acceptable salt herein which contains one or more stereocenters, each stereocenter can be independently, R or S. In some embodiments, any compound or pharmaceutically acceptable salt herein can have an enantiomeric excess or optical purity ranging from about 0% to 100%, or about: 5%, 10%, 15%, 20%, 25%, 30%, 35%, 40%, 50%, 55%, 60%, 65%, 79%, 75%, 80%, 85%, 90%, 95%, or 100% as determined by optical rotation of plane polarized light, for example at the sodium D-line. In some embodiments, any compound or pharmaceutically acceptable salt herein can have a diastereomeric excess or diastereomeric purity ranging from about 0% to 100%, or about: 5%, 10%, 15%, 20%, 25%, 30%, 35%, 40%, 50%, 55%, 60%, 65%, 79%, 75%, 80%, 85%, 90%, 95%, or 100% as determined by optical rotation of plane polarized light, for example at the sodium D-line.

In some embodiments, in the pharmaceutically acceptable salt of formula IV, X⁻ can be an anion selected from the group consisting of: chloride, bromide, sulphate, phosphate, acid phosphate, acetate, maleate, fumarate, lactate, tartrate, citrate, gluconate, and besylate. In some embodiments, in the pharmaceutically acceptable salt of formula IV, Z can be hydrogen (H). In some embodiments, in the pharmaceutically acceptable salt of formula IV, Z can be a pharmaceutically acceptable organic or inorganic cation.

In some embodiments, the pharmaceutically acceptable salt of formula IV can have the structure of formula V:

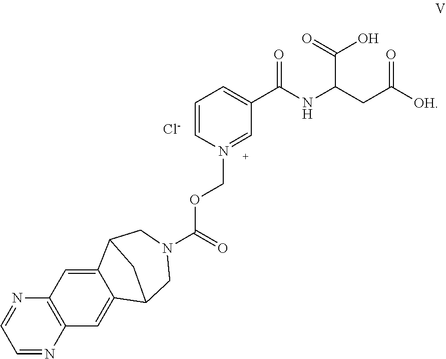

In some embodiments, the pharmaceutically acceptable salt of formula VI can have the structure of formula VI:

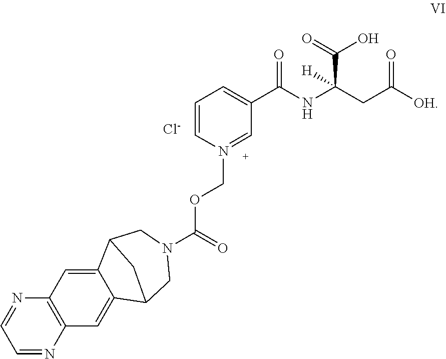

In some embodiments is provided a compound of formula VII or a pharmaceutically acceptable salt thereof:

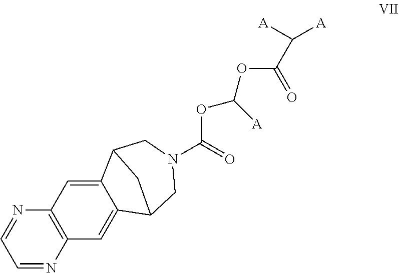

wherein each A, can be independently, a C1-C5 straight chain or branched chain alkyl group.

In some embodiments, in a compound or pharmaceutically acceptable salt thereof of formula VII, each A can be, independently, methyl.

In some embodiments is provide a compound of formula VIII or a pharmaceutically acceptable salt thereof:

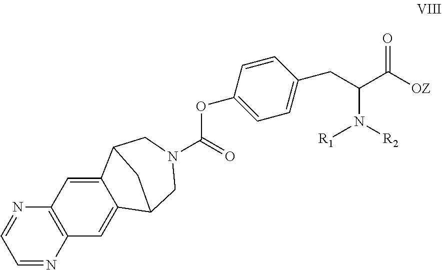

wherein R¹ and R² can be independently, hydrogen (H) or a C1-C5 branched or straight chain alkyl, or R¹ and R² together with the nitrogen atom to which they are attached can form a 5, 6, or 7 membered ring, and Z can be hydrogen (H) or a pharmaceutically acceptable cation.

In some embodiments, in a compound of formula VIII or a pharmaceutically acceptable salt thereof, R¹ and R² and Z can be hydrogen.

In some embodiments is provided a compound or pharmaceutically acceptable salt thereof of the following formula:

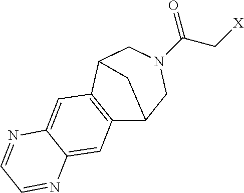

wherein X is OR₁, halogen, fluorine, chlorine, bromine, iodine, or NR₂R₃, wherein R₁ is hydrogen or an organic or inorganic cation or a C1-C10 straight or branched chain alkyl, and wherein R₂ and R₃ are each independently a C1-C10 straight or branched chain alkyl, or R₂ and R₃ together with the nitrogen atom to which they are attached form a 5-7 membered ring.

In some embodiments is provide a pharmaceutical composition comprising a compound of formulae I through VIII or pharmaceutically acceptable salt of formulae I through VIII and a pharmaceutically acceptable: excipient, diluent, carrier, or any combination thereof.

In some embodiments, the pharmaceutically acceptable carrier, diluent, or excipient, can include pharmaceutically acceptable excipients. As used herein, “excipient” can refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some embodiments, a pharmaceutically acceptable excipient can comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO Procipient, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some embodiments, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, a pharmaceutically acceptable excipient can comprise a carbohydrate. In some embodiments, the carbohydrate can comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

### Water as a Carrier

In some instances, a pharmaceutically acceptable carrier or diluent can comprise water. In some embodiments, the water can be sterile. In some embodiments, the water can contain a buffer, a carbohydrate, a salt, a pH adjuster, or any combination of these. Simple sugars such as mannitol, sucrose, glucose, trehelose may be added to inhibit peptide or polypeptide aggregation, in amounts from 1 to 50 mgs/ml. Citrate can be used as a buffer. In some instances, sodium chloride and phosphate salts may or may not be employed. Larger polysaccharides may also be used to enhance stability.

A carrier can refer to reagents, cells, compounds, materials, compositions, dosage forms, or any combination thereof that can be compatible with agents that can be administered therapeutically. In some cases, a carrier can be suitable for use in contact with a tissue of a subject. In some cases, a carrier may not have a toxicity, an irritation, an allergic response, or any combination thereof. A carrier that may be suitable for use can include a liquid, a solid material (e.g., a pill, or a suppository) or any combination thereof. In some cases, a carrier can be designed to resist degradation within the body (non-biodegradable) or they may be designed to degrade within the body (biodegradable). A biodegradable material can further be bioresorbable or bioabsorbable. In some cases, a biodegradable material can be degraded and eliminated from the body by conversion into other materials or breakdown and elimination through natural pathways.

### Representative Dosage Forms

In some embodiments, a composition or pharmaceutical composition can be in the form of a capsule, a tablet, a gummy, an oil, a liquid, an inhaled dosage form which can be a powder or a liquid delivered as an aerosol, a tincture, a lotion, a cream, a balm, a candy, a chocolate, a food, a drink, an oil, a suppository, a liquid for injection, which can be, for example, an intra venous liquid, an intra muscular liquid, or subcutaneous liquid; a syrup or any combination thereof.

In some embodiments, the pharmaceutical composition can be in unit dose form.

In some embodiments, a method of treating or preventing a disease or condition in a subject is provided, which can be a subject in need thereof, which can be a mammal, which can be a human subject, which can be a human subject in need thereof, comprising administering a compound or pharmaceutically acceptable salt of formulae I through VIII, of a pharmaceutical composition, to the human subject in an amount sufficient to treat the disease or condition. In some embodiments, the disease or condition can be tobacco use disorder, nicotine use disorder, smoking, Parkinson's disease, or any combination thereof. In some embodiments, the disease or condition comprises tobacco use disorder.

### Dosing Schedules and Amounts

In some embodiments, the administering can be once, twice, or three times per day; or once every other day; or once every third day. In some embodiments, the administering can be conducted for a period of a day, a week, a month, 6 months, a year, two years, three years, four years, five years, for life, or as needed. In some embodiments, the human or the subject or the mammal can be a male or a female. In some embodiments, the human or the subject or the mammal can be age 18 or older. In some embodiments, the administering can be oral or inhaled or intranasal or subterraneous or intramuscular or intravenous administrating. In some embodiments, a compound or pharmaceutically acceptable salt or the compound of formulae I through VIII can be administered in a dosage amount ranging from about 1 mg to about 20 mg, or about 1 mg to about 15 mg, or about 1 mg to about 10 mg, or about 1 mg to about 5 mg, or about 1 mg to about 3 mg.

In some instances, the amount of a compound or pharmaceutically acceptable salt thereof can be dosed in an amount ranging from about 0.0001 mg/kg of body weight of the subject to about 1000 g/kg or mg/kg of body weight of the subject or the subject in need thereof, the dosage can be, for example, based on mg of the pharmaceutically acceptable salt or compound, or a derivative thereof, per kg of subject body weight, can be about: 0.0001, 0.001, 0.01. 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 g/kg or mg/kg of subject body weight.

In some instances, the amount of the compound or pharmaceutically acceptable salt, can be dosed to the subject or subject in need thereof can range from 0.00001 mg to 1000 g; the dosage can be for example, about: 0.0001, 0.001, 0.01. 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 g or mg.

### Routes of Administration

In some embodiments, the pharmaceutical composition or compound or pharmaceutically acceptable salt thereof can be administered to the subject by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a transdermal route, or any combination thereof.

In some embodiments, the pharmaceutical composition or compound or pharmaceutically acceptable salt thereof can be formulated for oral administration. In some embodiments, the pharmaceutical composition can be in the form of a pill or a liquid. In some embodiments, a second therapy can be administered concurrently or consecutively with the pharmaceutical composition. In some embodiments, the second therapy is administered as part of the pharmaceutical composition. In some embodiments, the second therapy is administered separately from the pharmaceutical composition.

In some embodiments, a kit comprising a compound or a pharmaceutically acceptable salt of formulae I through VIII, or pharmaceutical composition containing one or more of these, and a container is provided.

In some embodiments, a method of making the kit, comprising placing a compound or a pharmaceutically acceptable salt of formulae I through formulae VIII, or the pharmaceutical composition containing these, in the container is provided.

In some embodiments, methods of making the compounds or pharmaceutically acceptable salts of formulae I through VIII, and also intermediate compounds and their salts useful for making these are provided.

In some embodiments, a compound or a pharmaceutically acceptable salt of formulae I through VIII, can have one or more unexpected superior characteristics including an unexpected superior: release profile, peak plasma concentration (Cmax), area under the curve (AUC) for 8, 16, or 24 hours, or infinity, or half-life when compared to administering a substantially similar amount of varenicline or a pharmaceutically acceptable salt thereof via the same route of administration.

### EXAMPLES

The following examples are included for illustrative purposes only and are not intended to limit the scope of the disclosure.

### Example 1

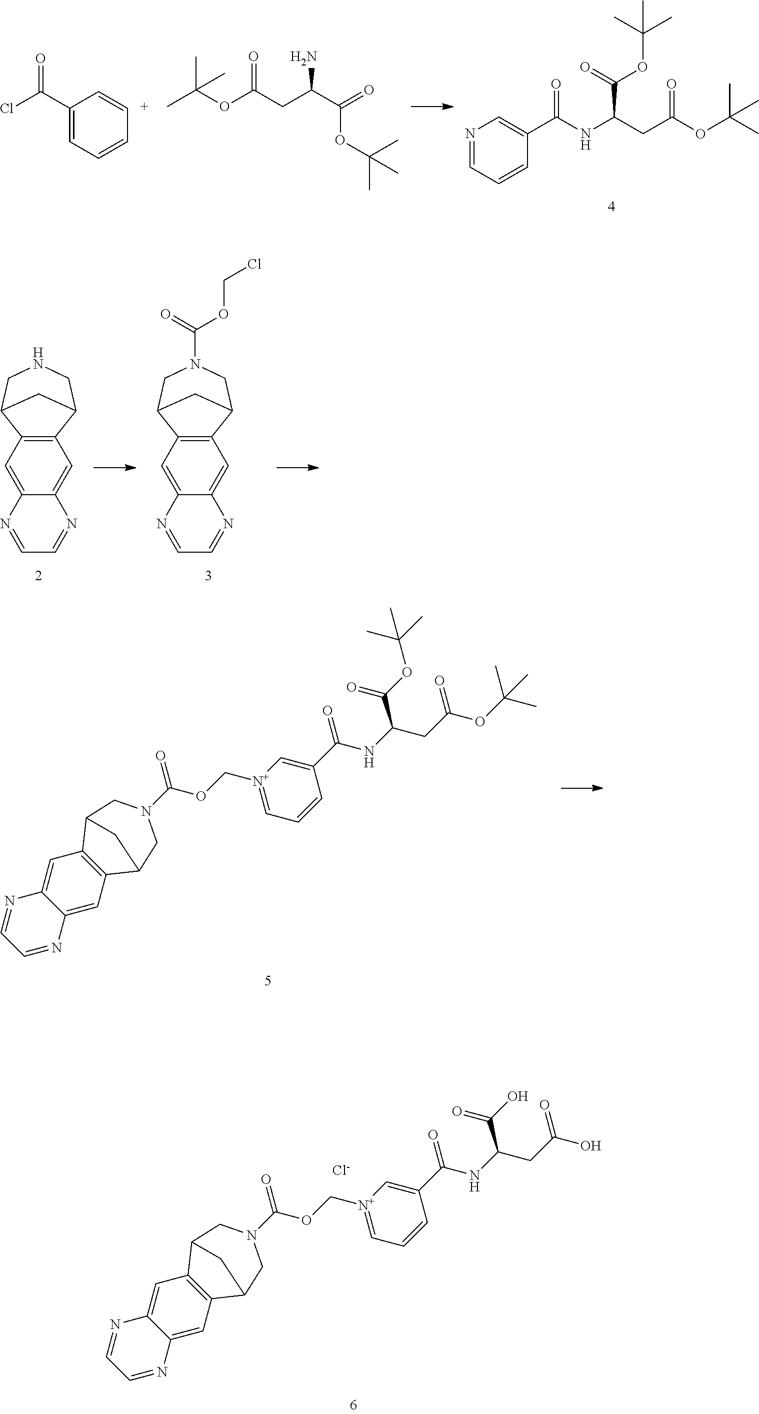

Synthesis of nicotinoyl-Asp(OtBu)-OtBu: 2; to H-Asp(OtBu)-OtBu·HCl (1.0 eq.) in dichloromethane (DCM) was added Et₃N (2.0 eq.). Nicotinoyl chloride hydrochloride (2.0 eq.) was added in portions (4 times, over 20 min.) in an ice-bath. After adding, the mixture was stirred for 1 h below 5° C. Water (30 mL) was added to quench the reaction, followed by DCM. The DCM layer was further washed with 5% NaHCO₃ and brine and dried over Na₂SO₄. The solvent was evaporated and the residue was purified by silica gel column chromatography (MeOH/DCM).

Synthesis of varenicline-N—CO₂CH₂—Cl, 3; varenicline (1.0 eq.) was purified and the initial synthesis carried out in DCM and cooled in an ice bath. 4-Dimethylaminopyridine (DMAP) (4.0 eq.) was added and the resulting mixture was stirred for 10 min. Chloromethyl chloroformate (2.5 eq.) in DCM was added slowly. The ice bath was removed and the reaction was stirred for 5 h at room temperature. Ethyl acetate was added, followed by water (20 mL) to quench the reaction. The ethyl acetate layer was separated and washed with 1N HCl and brine and dried over anhydrous sodium sulfate. The solvent was evaporated and the residue was purified by silica gel column chromatography.

Synthesis of varenicline-N—CO₂CH₂-nicotinoyl-Asp(OtBu)-OtBu, 5; The chloromethyl carbamate of varenicline (1.5 eq.) and nicotinoyl-Asp(OtBu)-OtBu, 2 (1.0 eq.) in acetonitrile were heated for 24 h at 70° C. The solvent was evaporated. The residue was purified by silica gel column chromatography.

Synthesis of 6 chloride; varenicline-N—CO₂CH₂— nicotinoyl-Asp(OtBu)-OtBu chloride in 4N HCl/dioxane was stirred for 3 h. The solvent was evaporated. The residue was coevaporated with DCM), then dissolved in DCM (4 mL) and TBME (25 mL) was added. The resulting solid was collected, washed with TBME (4×2 mL) and dried in vacuum. Yield resulted in a solid 6 chloride.

### Example 2

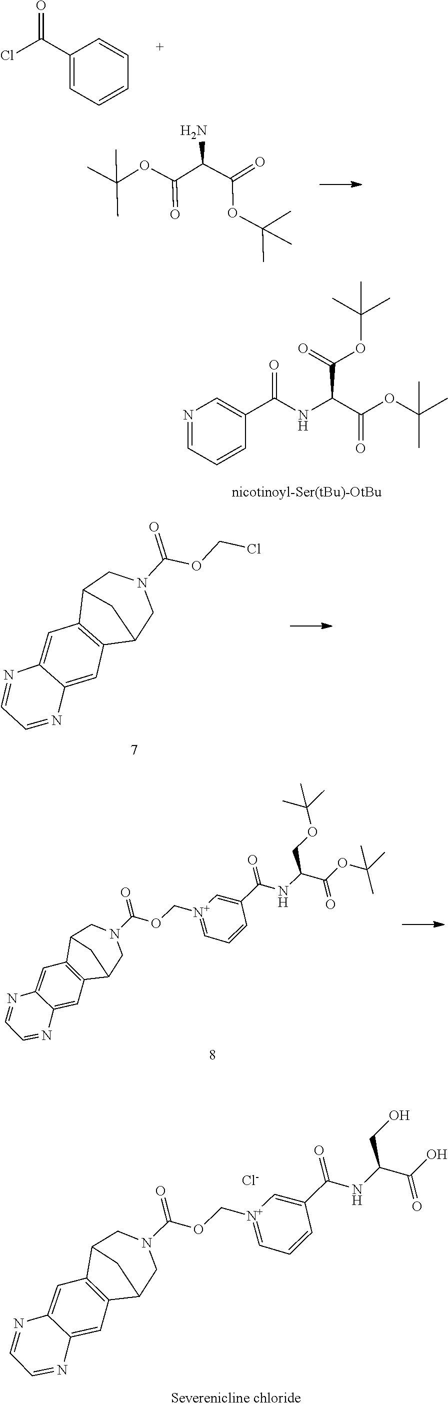

### Synthesis of Servarenicline Chloride

Synthesis of nicotinoyl-Ser(tBu)-OtBu; to H-SER(TBU)-OTBU (1.0 eq.) in DCM was added Et₃N (2.0 eq.). Nicotinoyl chloride hydrochloride (2.0 eq.) was added in portions (4 times, over 20 min.) in an ice-bath. After adding, the mixture was stirred for 1 h below 5° C. Water (30 mL) was added to quench the reaction, followed by DCM. The DCM layer was further washed with 5% NaHCO₃ and brine and dried over Na₂SO₄. The solvent was evaporated and the residue was purified by silica gel column chromatography (MeOH/DCM).

Synthesis of varenicline-N—CO₂CH₂—Cl, 7; varenicline (1.0 eq.) in DCM and cooled in an ice bath. 4-Dimethylaminopyridine (DMAP) (4.0 eq.) was added and the resulting mixture was stirred for 10 min. Chloromethyl chloroformate (2.5 eq.) in DCM was added slowly. The ice bath was removed and the reaction was stirred for 5 h at room temperature. Ethyl acetate was added, followed by water (20 mL) to quench the reaction. The ethyl acetate layer was separated and washed with 1N HCl and brine and dried over anhydrous sodium sulfate. The solvent was evaporated and the residue was purified by silica gel column chromatography.

Synthesis of varenicline-N—CO₂CH₂— nicotinoyl-Ser(tBu)-OtBu, 8; The chloromethyl carbamate of varenicline (1.5 eq.) and nicotinoyl-Ser(tBu)-OtBu (1.0 eq.) in acetonitrile were heated for 24 h at 70° C. The solvent was evaporated. The residue was purified by silica gel column chromatography.

Synthesis of Servareniclin chloride: varenicline-N—CO₂CH₂— nicotinoyl-Ser(tBu)-OtBu chloride in 4N HCl/dioxane was stirred for 3 h. The solvent was evaporated. The residue was coevaporated with DCM), then dissolved in DCM (4 mL) and TBME (25 mL) was added. The resulting solid was collected, washed with TBME (4×2 mL) and dried in vacuum. Yield resulted in a solid.

### Example 3 Synthesis of Varenicline-Enacarbil

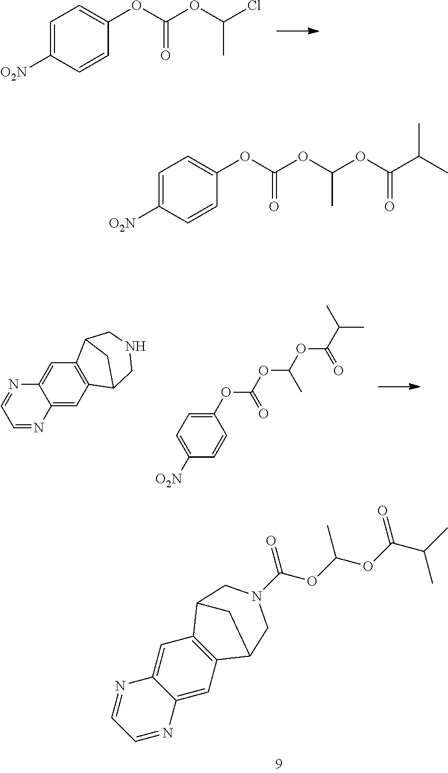 [SMILES: CC(Cl)OC(=O)Oc1ccc([N+](=O)[O-])cc1 ; CC(OC(=O)Oc1ccc([N+](=O)[O-])cc1)OC(=O)C(C)C ; c1cnc2cc3c(cc2n1)C1CNCC3C1 ; CC(OC(=O)Oc1ccc([N+](=O)[O-])cc1)OC(=O)C(C)C ; CC(OC(=O)C(C)C)OC(=O)N1CC2CC(C1)c1cc3nccnc3cc12]

Synthesis of 1-(isobutyryloxy)ethyl 4-nitrophenyl carbonate; zinc oxide was added (1 g, 12 mmol) to a solution of toluene (40 ml) and isobutyric acid (10 ml) and refluxed for an hour to remove the water created in this process by Dean-Stark apparatus. After lowering the temperature to 60° C., 1-1-chloroethyl 4-nitrophenyl carbonate (1 g, 4 mmol) and NaI (1 g, 6.4 mmol) were added to the reaction mixture. The mixture was stirred at 60° C. for 24 hours. On the following day, evaporate the reaction mixture was evaporated to residue then the residue was dissolved in EtOAc, the organic solution was washed with saturated NaHCO₃ solution and then with brine. the organic layer was separated and dried over MgSO₄. The dried organic layer was evaporated to obtain 1-(isobutyryloxy)ethyl 4-nitrophenyl carbonate.

Varenicline enacarbil, 9; To varenicline (1.0 eq. 5.75 g, 0.03 mol) in toluene (50 ml) was added chlorotrimethyl silane (2.0 eq.) and tributyl amine (2.0 eq.) which was then stirred at room temperature to obtain clear solution. Then 1-(isobutyryloxy)ethyl 4-nitrophenyl carbonate (1.0 eq.) in toluene (20 ml) was added to the reaction mixture which was stirred at room temperature for 24 hours. After the completion of reaction, reaction mixture was wash with 1N HCl (150 ml) and water (2×100 ml). The organic layer was dried over MgSO₄, evaporated, and purified by silica gel column chromatography to obtain desired product.

### Example 4 Synthesis of Varenicline-CBT

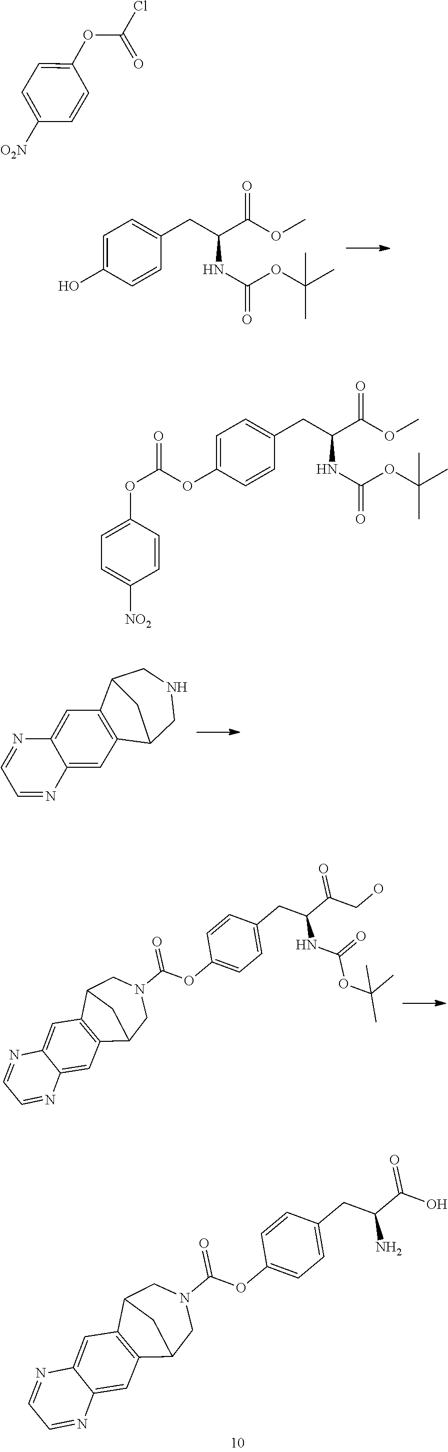 [SMILES: O=C(Cl)Oc1ccc([N+](=O)[O-])cc1 ; COC(=O)[C@H](Cc1ccc(O)cc1)NC(=O)OC(C)(C)C ; COC(=O)[C@H](Cc1ccc(OC(=O)Oc2ccc([N+](=O)[O-])cc2)cc1)NC(=O)OC(C)(C)C ; c1cnc2cc3c(cc2n1)C1CNCC3C1 ; CC(C)(C)OC(=O)N[C@@H](Cc1ccc(OC(=O)N2CC3CC(C2)c2cc4nccnc4cc23)cc1)C(=O)CO ; N[C@@H](Cc1ccc(OC(=O)N2CC3CC(C2)c2cc4nccnc4cc23)cc1)C(=O)O]

Synthesis of 4-Nitrophenyl Boc-Tyr-OMe; the mixture of Boc-Tyr-OMe, 4-Nitrophenyl Chloroformate, and DIEA in DCM was stirred for 18 hours at room temperature, After the completion of reaction, organic layer was wash water, dried over MgSO₄, and evaporated. Crude product was used as it is for the following step.

Synthesis Varenicline-CBT, 10; A solution of varenicline in DMF was added to a mixture of 4-Nitrophenyl Boc-Tyr-OMe and diisopropyl ethyl amine (DIEA) in N,N-dimethyl formamide (DMF) then stirred at room temperature for 3 hours. After the completion of reaction, added EtOAc to reaction mixture then washed with 1N HCl (150 ml) and water (2×100 ml). The organic layer was dried over MgSO₄, evaporated, and purified by silica gel column chromatography to obtain desired product. The purified product was stirred in 6N HCl/dioxane for 24 h at 40° C. The solvent was evaporated. The residue was coevaporated with DCM, then dissolved in DCM (4 mL) and TBME (25 mL) was added. The resulting solid was collected, washed with TBME (4×2 mL) and dried in vacuum. Yield a solid

### Example 5

The compounds or pharmaceutically acceptable salts of formulae I through VIII are individually formulated, individually, into dosage forms, in therapeutically effective amounts, and are administered individually to individual subjects. The compounds or pharmaceutically acceptable salts will optionally demonstrate extended release profiles and will show blood plasma concentrations sufficient to be therapeutically effective.

### Example 6

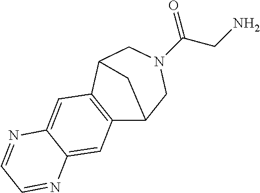 [SMILES: NCC(=O)N1CC2CC(C1)c1cc3nccnc3cc12]

### Synthesis of Varenicline-Glycinamide

Chloromethyl carbamate of varenicline; a solution of chloromethyl chloroformate in DCM (dichloromethane) was added into the mixture of varenicline, 4-DMAP, and DCM at ice-bath temp, allowed to warm, and stirred for 5 hours at room temperature. Varenicline-Glycinamide; a mixture of ammonium hydroxide and chloromethyl carbamate of varenicline at room temp for 3 hours.

### Example 7

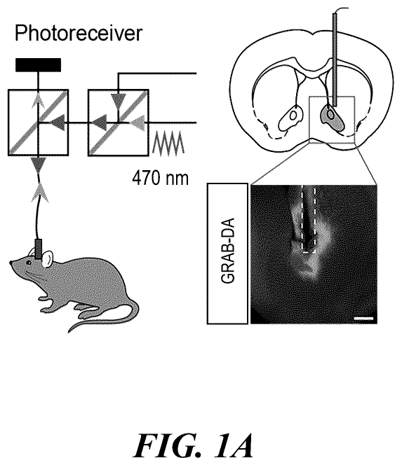

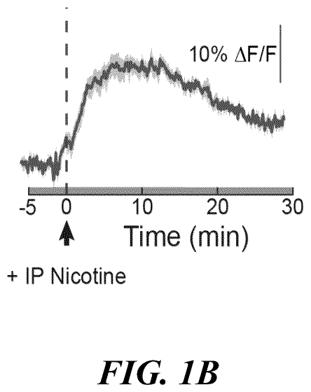

The ability of varenicline to decrease nicotine use comes from the ability to block nicotine-induced dopamine signaling in the striatum. Real-time sensing of dopamine levels in awake and behaving mice was measured using a fluorescent dopamine sensor (GRAB-DA) and fluorometric measurements of that sensor (Fiber Photometry). FIG. 1A shows the experimental placement of the fiber in the striatum and an exemplary fluorescent image produced during the experiment. FIG. 1B shows an exemplary fluorescent response curve preceding and following an intraperitoneal administration of nicotine.

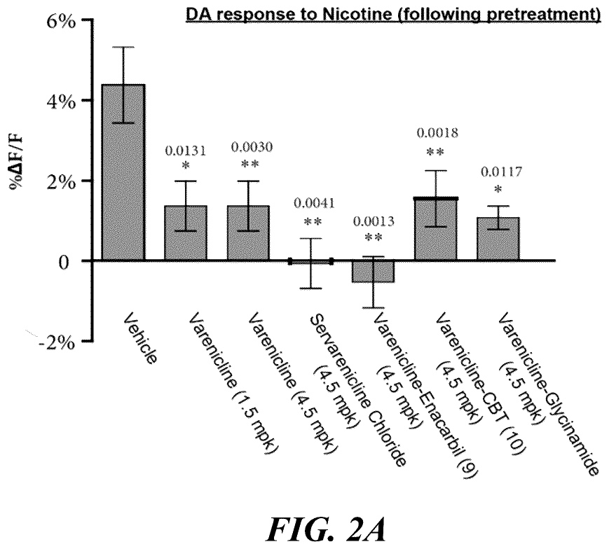

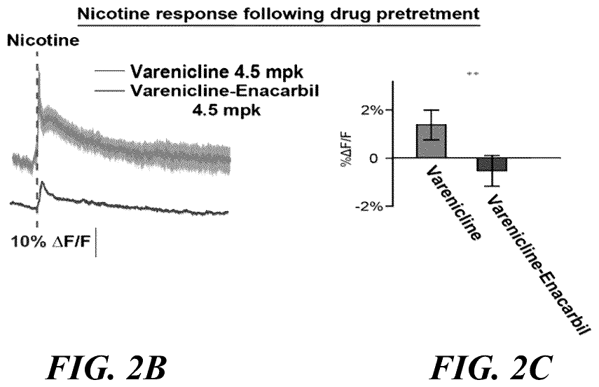

It was found that the varenicline derivatives designed herein have equal or greater efficacy than varenicline at reducing the nicotine-evoked release of dopamine (FIG. 2A). These data demonstrated that regardless the dose of varenicline, the response to nicotine 2 hours later is the same. Servarenicline Chloride and Varenicline Encarbil (FIGS. 2B-2C) showed better efficacy when compared directly to varenicline, but all the derivatives worked at least as well as varenicline.

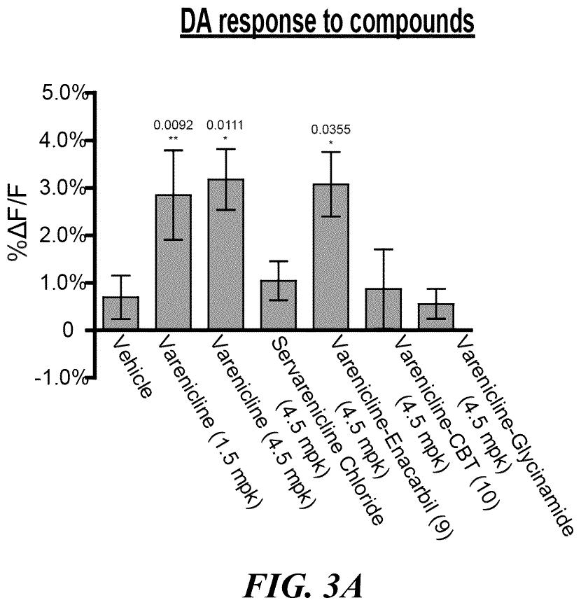

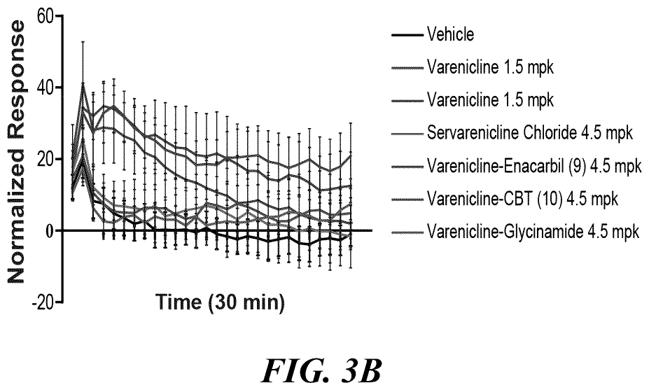

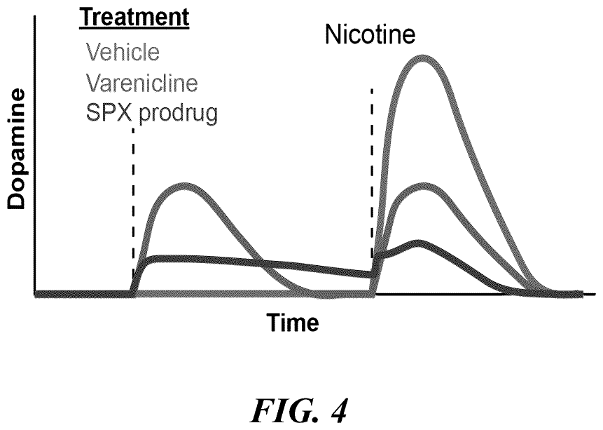

Varenicline increased striatal dopamine levels after administration. This increase is thought to potentially reduce nicotine craving, but may also be related to the unwanted side effects of varenicline. It was found that the varenicline based molecules described herein have differing abilities to induce dopamine following oral administration (FIGS. 3A-3B). Some molecules (e.g., Servarenicline Chloride, Varenicline-CBT, or Varenicline Glycinamide) do not lead to dopamine release, while others (Varenicline-Encarbil) mimic the effects of varenicline.

This suite of compounds provided two new advantages. First, a set of molecules that mimicked the response of varenicline, but were engineered to last longer. Second, molecules that smoothed the dopamine response, preventing the surge of dopamine normally seen by the partial agonist function of varenicline.

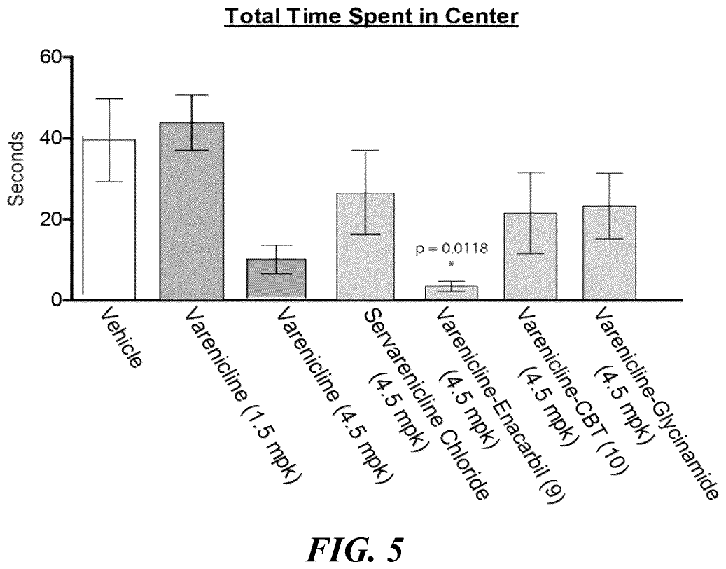

Varenicline also influenced the mobility and anxiety-like behavior in mice. In the open field assay (FIG. 5), animals exhibited anxiety-like behavior in avoidance of open areas (dramatically reduced time in the middle). This effect was not observed for Servarenicline Chloride, Varenicline-CBT, or Varenicline Glycinamide, suggesting that these varenicline derivatives may have a different side effect profile.

While preferred embodiments have been shown and described herein, such embodiments are provided by way of example only. Numerous variations, changes, and substitutions will now occur to those skilled in the art without departing from the disclosure herein. It should be understood that various alternatives to the embodiments described herein may be employed.

What is claimed is:

1. A pharmaceutically acceptable salt of formula I: 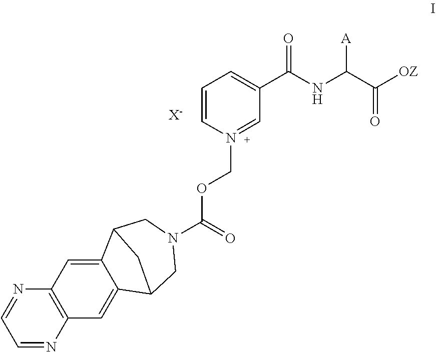
wherein, independently, X⁻ is a pharmaceutically acceptable anion, A is a C1-C10 alkyl or branched alkyl group bearing a hydroxyl group, and Z is hydrogen or a pharmaceutically acceptable cation.

2. The pharmaceutically acceptable salt of formula I of claim 1, wherein X⁻ is a pharmaceutically acceptable anion selected from the group consisting of: chloride, bromide, sulphate, phosphate, acid phosphate, acetate, maleate, fumarate, lactate, tartrate, citrate, gluconate, and besylate.

3. The pharmaceutically acceptable salt of formula I of claim 1, wherein Z is H.

4. The pharmaceutically acceptable salt of formula I of claim 1, wherein Z is a pharmaceutically acceptable organic or inorganic cation.

5. The pharmaceutically acceptable salt of formula I of claim 1, having the structure of formula II:

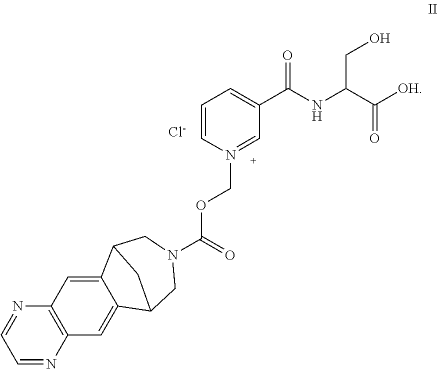

6. The pharmaceutically acceptable salt of formula I of claim 1, having the structure of formula III:

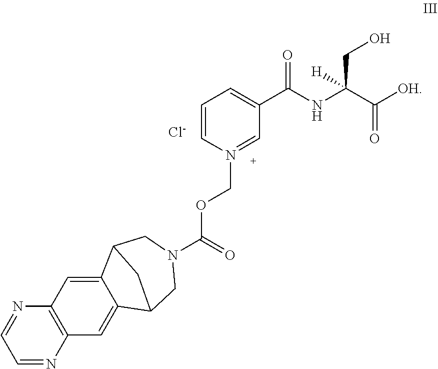

7. A pharmaceutically acceptable salt of a compound of formula IV: 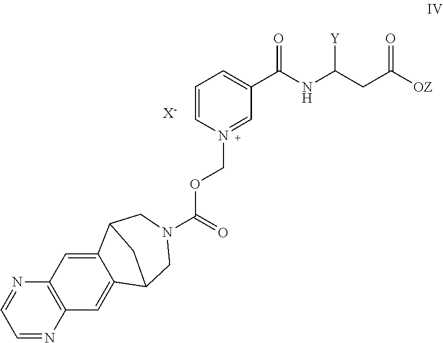
wherein, independently, X⁻ is a pharmaceutically acceptable anion, Y is a C1-C10 straight chain or branched chain alkyl group bearing a carboxylic acid group or a pharmaceutically acceptable salt thereof, and Z is hydrogen or a pharmaceutically acceptable cation.

8. The pharmaceutically acceptable salt formula IV of claim 7, wherein X⁻ is a pharmaceutically acceptable anion selected from the group consisting of: chloride, bromide, sulphate, phosphate, acid phosphate, acetate, maleate, fumarate, lactate, tartrate, citrate, gluconate, and besylate.

9. The pharmaceutically acceptable salt of formula IV of claim 7, wherein Z is H.

10. The pharmaceutically acceptable salt of formula IV of claim 7, wherein Z is a pharmaceutically acceptable organic or inorganic cation.

11. The pharmaceutically acceptable salt formula IV of claim 7, having the structure of formula V:

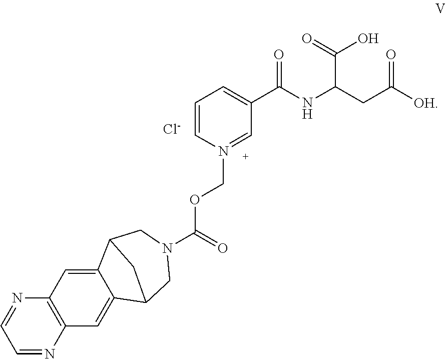

12. The pharmaceutically acceptable salt of formula IV of claim 7, having the structure of formula VI:

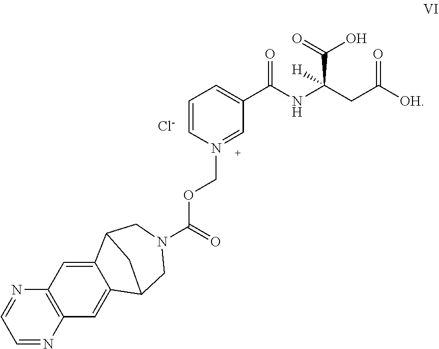

13. A pharmaceutically acceptable salt of formula IA: 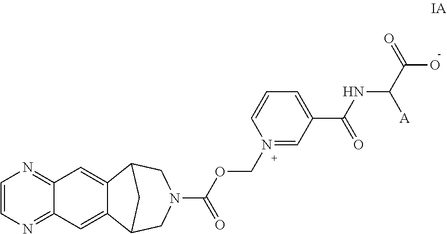
wherein A is a C1-C10 alkyl or branched alkyl group bearing a hydroxyl group or a C1-C10 straight chain or branched chain alkyl group bearing a carboxylic acid group or a pharmaceutically acceptable salt thereof.

14. A compound of formula VII or a pharmaceutically acceptable salt thereof: 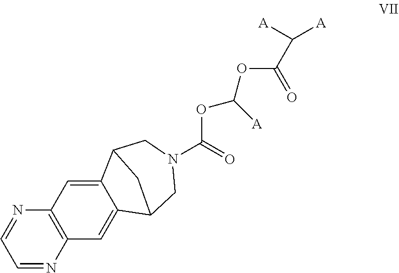
wherein each A, is independently, a C1-C5 straight chain or branched chain group.

15. The compound or pharmaceutically acceptable salt thereof of formula VII of claim 14, wherein each A is, independently, a methyl group.

16. A compound of formula VIII or a pharmaceutically acceptable salt thereof: 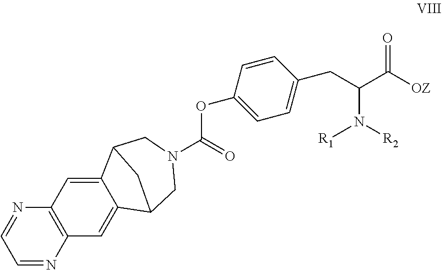
wherein R¹ and R² are independently, hydrogen or a C1-C5 branched or straight chain alkyl, or R¹ and R² together with a nitrogen atom to which they are attached form a 5, 6, or 7 membered ring, and Z is hydrogen or a pharmaceutically acceptable cation.

17. The compound of formula VIII or a pharmaceutically acceptable salt thereof of claim 16, wherein R¹ and R² are hydrogen, and wherein Z is hydrogen.

18. A pharmaceutical composition comprising the compound or pharmaceutically acceptable salt of claim 1; and a pharmaceutically acceptable: excipient, diluent, or carrier.

19. A method of treating or preventing a disease or condition in a human subject in need thereof, comprising administering the compound or pharmaceutically acceptable salt thereof of claim 1, to the human subject in an amount sufficient to treat the disease or condition.

20. A kit comprising the compound or its pharmaceutically acceptable salt of any one of claim 1, and a container.
